(19) United States
(12) Patent Application Publication
(10) Pub. No.: US 2014/0066745 A1
(43) Pub. Date: Mar. 6, 2014

(54) MEDICAL IMAGING SYSTEM AND METHOD USING DIFFERENT COMPUTERS FOR CONTROL OF DATA ACQUISITION AND FOR IMAGE RECONSTRUCTION

(71) Applicant: Swen Campagna, Engelthal (DE)

(72) Inventor: Swen Campagna, Engelthal (DE)

(21) Appl. No.: 14/018,796

(22) Filed: Sep. 5, 2013

(30) Foreign Application Priority Data
Sep. 5, 2012 (DE) 102012215721.2

(51) Int. Cl. A61B 5/055

(52) U.S. Cl. CPC A61B 5/055
USPC 600/407

(57) ABSTRACT

An imaging system, includes an imaging installation or an imaging scanner to generate raw data; a first computer that is connected with the imaging installation or the imaging scanner for controlling the imaging installation or the imaging scanner, a memory to store the generated raw data; and a second computer to calculate an image from the stored raw data.

### BACKGROUND OF THE INVENTION

1. Field of the Invention

The present invention is in the fields of medical engineering and information technology, and in particular concerns imaging methods and systems, for example diagnostic magnetic resonance (MR) methods and MR systems or other installations.

2. Description of the Prior Art

The conventional diagnostic MR system 10 shown schematically in FIG. 1 has an MR computer 100 and an MR installation or MR scanner 200.

The MR computer 100 has a communication interface 105₁, a processor 110, working memory 120, an input device 160, an output device 170 and a memory 180. The working memory 120 stores an operating system 130, an application program 140 and data (for example a sequence 150). The input device 160 serves to accept inputs of a user, and the output device 170 serves to output information to the user. The memory 180 serves to store MR data 190, for example raw MR data and MR image data. With a communication interface 105₁, the MR computer 100 is connected via a connection 310 with the MR installation 200 and serves to receive, process and execute a measurement task. For this, the MR computer 100 loads the sequence 150 and generates instructions for the MR installation 200. The MR computer 100 can be executed as a high-performance workstation, for example.

The MR installation/MR scanner 200 includes devices—for example a gradient arrangement (gradient array) with gradient amplifier and gradients, a transmission unit (transmitter unit, TX unit) with radio-frequency (RF) amplifier and radio-frequency coil and a receiver unit (RX unit)—and is controlled by the MR computer 100 via the instructions. The instructions serve to control the gradients of the gradient array, to control the transmitter unit and to process radio-frequency signals in the transmitter unit, or to control the receiver unit and digitize RF reception signals in said receiver unit.

In the operation of the MR system 10, the measurement workflow is controlled during the examination via the sequence, which exactly establishes points in time of gradient activity, radio-frequency pulses and measurements.

MR images are calculated or reconstructed from MR measurements in the form of MR signals or, respectively, MR data (for example raw data). The computing cost for the image calculation or, respectively, image reconstruction is thereby dependent on the calculation or, respectively, reconstruction method and a data set of an MR measurement, and a resulting computing time is dependent on a computing power of the MR computer 100. While a simple reconstruction method can calculate MR images nearly without delay, a complicated reconstruction method for qualitatively high-grade MR images requires a significantly longer computing time in the range from a few minutes up to multiple hours, even if it is executed at a very high-grade computer with very fast processor.

In such a conventional system, for the calculation of an MR image within the scope of a clinical routine it is required to compromise between the quality of the MR image and the computing time resulting from this.

For this purpose, an MR measurement must be parameterized so that the MR system 10 calculates or reconstructs an MR image at a sufficiently good quality given simultaneously acceptable computing time.

Alternatively, an MR measurement could initially calculate a simple MR image using a simple calculation method for prompt monitoring of the received MR signals, in order to identify possible errors that occur as movement artifacts, for example, and possibly to be able to implement a repeat MR measurement immediately as long as a patient to be examined is still located in the MR installation 200. After conclusion of the MR measurement, within the scope of a retrospective reconstruction (retro recon) the MR system 10 could calculate—using a high-grade calculation method—a high-grade MR image from the raw MR data that have merely been cached during the MR measurement.

A disadvantage of the MR system 10 according to the prior art is the dependency of the computing time on the quality of the MR images or the compromise that is required and the repeated calculation of an MR image initially (during the MR measurement) using the simple calculation method and subsequently (after conclusion of the MR measurement) using the high-grade calculation method or the temporal occupation of the MR system 10 that results from this beyond the actual MR measurement.

### SUMMARY OF THE INVENTION

An object of the present invention is to provide an improved MR system with regard to the aforementioned disadvantages of conventional systems.

In the following, the achievement of the object is described with reference to a control device. The advantages and/or alternative embodiments that are mentioned also apply to an imaging system, a method, or a non-transitory, computer-readable storage medium encoded with programming instructions, that are also within the scope of the invention.

An imaging or image-processing system is a system, such as an electronic and/or information technology system, for the acquisition, processing, evaluation and/or storage of image information in the form of image data. For example, acoustic methods such as ultrasound (US); emission methods such as emission computer tomography (ECT) and positron emission tomography (PET); optical methods; radiological methods such as x-ray tomography and computer tomography (CT); magnetic resonance tomography (MR), or combined methods can be used to acquire the image information. The image-processing system can deliver 2-dimensional (2D) or multidimensional (such as 3-dimensional (3D) or 4-dimensional (4D)) image data that can be stored and/or processed in different formats. The image-processing system can be used in diagnostics, for example in medical diagnostics.

A processor (central processing unit, CPU) is, for example, a microprocessor or digital signal processor (DSP). The processor, which is controlled by the program that can be split into a number of program modules, writes data into the memory, reads data from the memory and processes the data. For example, the processor can also be executed as an (application) field programmable (logic) gate array (FPGA).

For example, the memory can be a read-only memory (ROM) such as an electrically erasable programmable read-only memory (EEPROM) or Flash EEPROM, read/write memory (random access memory, RAM), and disk storage such as fixed disk storage. The memory can be used to store a program (for example an operating system or an application program) and/or data (in particular image data, instruction data, configuration data, parameter data, program data and sequence data.

An operating system includes computer programs that administer system resources (hardware components) of a computer (for example memory such as working memory and fixed disks, input and output devices such as interfaces) and provides application programs. The operating system thus forms an interface between the system resources and the application programs.

One aspect of the invention concerns an imaging system that has an imaging installation or an imaging scanner for the generation of raw data; a first computer that is connected with the imaging installation or the imaging scanner for control of the imaging installation or of the imaging scanner, a memory to store the generated raw data, and a second computer to calculate an image from the stored raw data.

In this aspect of the invention, the raw data are removed from the imaging scanner without preventing the first computer from itself calculating or reconstructing images (for example simple images with higher priority) from these, such that the second computer can calculate or reconstruct images (for example complicated images with lower priority) while the first computer continues to control and monitor the imaging installation or the imaging scanner and the imaging installation or the imaging scanner continues to generate raw data.

A further aspect of the invention concerns an imaging system, furthermore has a network (for example the Internet) that is arranged between the memory and the second computer for the transfer of the stored raw data from the memory to the second computer.

A further aspect of the invention concerns an imaging system, wherein the second computer is executed as a workstation, virtual machine or computer in the cloud.

A calculation or, respectively, reconstruction of qualitatively high-grade images with low priority in the cloud is enabled via this aspect of the invention.

A further aspect of the invention concerns an imaging system, wherein the second computer calculates the image using a complicated calculation method.

A further aspect of the invention concerns an imaging system, wherein the first computer calculates an additional image from the raw data, wherein the first computer calculates the additional image using a simple calculation method.

A further aspect of the invention concerns an imaging system, wherein the memory is connected with the first computer.

A further aspect of the invention concerns an imaging system, wherein the memory is arranged in the first computer.

A further aspect of the invention concerns an imaging system, wherein the memory is arranged in the second computer.

A further aspect of the invention concerns an imaging system, wherein the memory second computer is connected with memory.

A further aspect of the invention concerns a computer-implemented method of an imaging system, that includes controlling an imaging installation or an imaging scanner that generates the raw data, with a first computer that is connected with the imaging installation or the imaging scanner storage of the generated raw data in a memory, and calculate an image from the stored raw data with a second computer.

A further aspect of the invention concerns a method, furthermore including transfer of the stored raw data from the memory to the second computer via a network (for example the Internet) that is arranged between the memory and the second computer.

A further aspect of the invention concerns a method, wherein the memory is connected with the first computer.

A further aspect of the invention concerns a method, wherein the memory is arranged in the first computer.

A further aspect of the invention concerns a method, wherein the memory is arranged in the second computer.

A further aspect of the invention concerns a method, wherein the second computer is connected with the memory.

The present invention also encompasses a non-transitory, computer-readable data storage medium encoded with programming instructions that, when the storage medium is loaded into respective computers of a medical imaging system, caused the computers to execute the method according to the invention, as described above.

A further aspect of the invention concerns a control device that has a calculation module configured to calculate an image from the raw data, using a simple calculation method.

A further aspect of the invention concerns a control device that is connected with the aforementioned memory.

A further aspect of the invention concerns a control device that has the aforementioned memory.

It is within the scope of the invention to execute the method steps in an order that differs from the order described above. Instead of relaying the information coming from the transmitting component directly to the receiving component, it is also alternatively possible, for example, to initially conclude the reception of the information from the transmitting component and to only then send the information to the receiving component. In a further embodiment, the method steps can also be interleaved with one another.

Moreover, it is possible that individual segments of the method described in the preceding can be fashioned as individual salable units, and remaining segments of the method can be fashioned as other salable units. The method according to the invention can therefore be executed as a distributed system at different computer-based instances (for example client/server instances). For example, it is possible that a module to include different sub-modules that, for example, are implemented in part at the measurement system, in part at the reconstruction system and/or in part at other computer-based instances.

### BRIEF DESCRIPTION OF THE DRAWINGS

FIG. 1 schematically illustrates a known MR system.

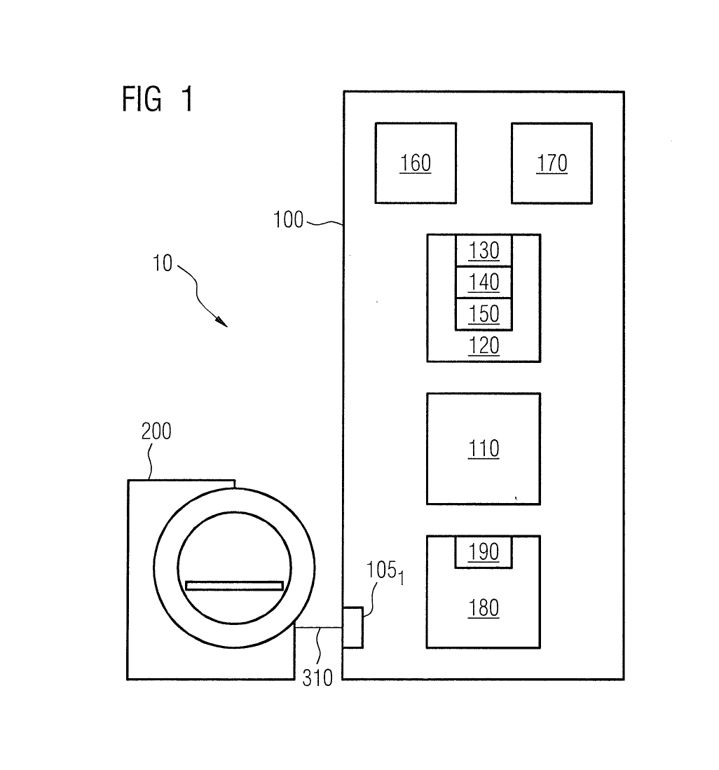

FIG. 2 schematically illustrates an MR system according to a preferred embodiment of the invention.

### DESCRIPTION OF THE PREFERRED EMBODIMENTS

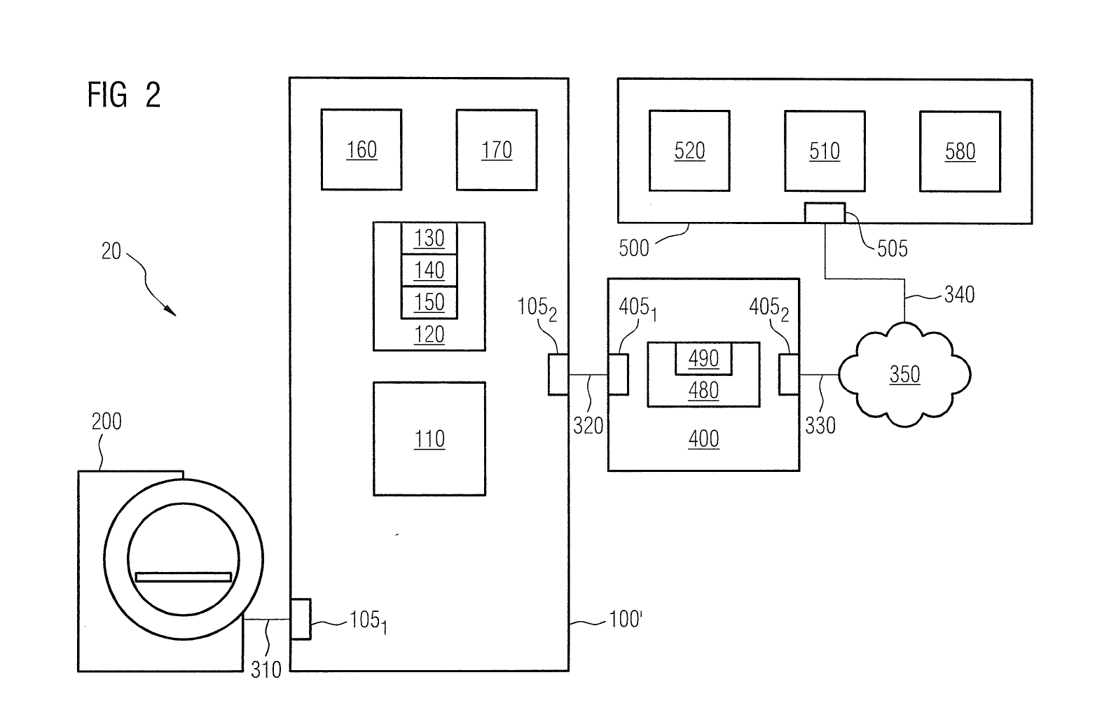

FIG. 2 is a schematic representation of an MR system 20 according to a preferred embodiment of the invention, that includes an MR computer 100′, an MR installation or an MR scanner 200, an MR memory 400, and a computer 500. The MR system 20 can furthermore include a network 350.

The MR computer 100′ comprises communication interfaces 105₁-105₂; a processor 110; working memory 120; an input device 160; and an output device 170. The working memory 120 stores an operating system 130, an application program 140 and data 150, for example MR measurement data and MR image data. The MR computer 100′ can be executed as a high-performance workstation, for example.

The MR computer 100′ can serve for interaction of the user or, respectively, operator with the MR system 20, for example to plan an examination of a patient by means of the MR system 20 and to receive a parameterized measurement task. For interaction with the user, the MR computer 100′ has a user interface, for example a graphical user interface (GUI). The input device 160 serves to receive inputs of a user or operator and can be embodied as a keyboard or computer mouse, for example. The output device 170 serves to output information to the user or operator and, for example, can be executed as a monitor.

Furthermore, the MR computer 100′ can serve to process and execute the measurement task. For this, the MR computer 100′ can, for example, load the sequence in the form of a freely programmable binary program and execute it in order to promptly generate instructions for the MR installation 200 with an actual execution point in time.

Furthermore, the MR computer 100′ can realize the instructions and, for example, serve for general communication tasks, such as to control gradients of a gradient array, to control a transmitter unit (TX unit) and to process radio-frequency signals in the transmitter unit, or to control a receiver unit (RX unit) and digitize radio-frequency (RF) reception signals in the receiver unit. For this purpose, for example, the MR computer 100′ can comprise digital signal processors (DSPs) or (application) field programmable (logic) gate arrays (FPGAs). The MR computer 100′ can furthermore include a computer, for example a measurement and control computer (measurement and reconstruction computer) or a measurement and control system (measurement and reconstruction system). The MR computer 100′ serves to realize the instructions that must be executed exactly at a desired or required point in time.

The MR computer 100′ can thus serve as an MR measurement console, MR measurement monitoring computer and MR controller.

The MR installation/the MR scanner 200 includes devices, such as the gradient array with gradient amplifier and the gradients, the transmitter unit with radio-frequency amplifier and radio-frequency coil and the receiver unit, and is connected via the connector 310 with the communication interface 105₁ of the MR computer 100′, and is controlled by the MR computer 100′.

The MR memory 400 comprises communication interfaces 405₁-405₂ and memory 480. With its communication interface 405₁, the MR memory 400 is connected via a connection 320 with the communication interface 105₂ of the MR computer 100′. The memory 480 can be a disk storage, for example hard disk storage such as a redundant array of independent disks (RAID) or network attached storage (NAS), and serves to store MR data 190 (for example MR signals and raw MR data) that are generated during an MR measurement and/or MR image data or MR images that are calculated or, respectively, reconstructed from these. The memory 400 can be designed as a computer, for example as a high-performance workstation or virtual machine.

The computer 500 comprises a communication interface 505, a processor 510 and working memory 120. Furthermore, the computer 500 can include a memory 580. for example, the computer 500 can be designed as a high-performance workstation or virtual machine and/or be outsourced with other computer to a server or the cloud. With its communication interface 505, the computer 500 is connected—directly or via a network 350, for example a wireless network or wired network, such as the Internet—via a connection 330, 340 with the communication interface 405₂ of the MR memory 400, and serves to execute image calculations or, respectively, image reconstructions from the MR data.

In operation of the MR system 20, the measurement workflow is controlled during the examination via the sequence 150, which exactly establishes points in time of gradient activity, radio-frequency pulses and measurements. Events in the form of physiological signals, for example, can be dynamically reacted to, and the measurement workflow can be adapted accordingly.

MR images (for example simple MR images and qualitatively high-grade MR images) are calculated or, respectively, reconstructed from MR measurements in the form of MR signals or, respectively, MR data (raw MR data, for example).

A simple MR image is initially calculated as a preliminary MR image using a simple calculation method or, respectively, reconstruction method, for example for prompt monitoring of the received MR signals. Possible errors (for example that occur as movement artifacts) can thereby be identified and, given an error, a repeat MR measurement can possibly be implemented immediately as long as a patient to be examined is still located in the MR installation 200. The image calculation or image reconstruction thereby takes place using the simple calculation method or, respectively, reconstruction method via the MR computer 100′, and the resulting computing time is dependent on the computing power of the MR computer 100′.

Since the MR computer 100′ executes a simple calculation method or, respectively, reconstruction method, the MR images are calculated nearly without delay, and a time cost for the early monitoring is shortened.

The received MR signals are stored as raw MR data outside of the MR computer 100′, for example in the memory 480 of the MR memory 400. The raw MR data can thereby be accessed at any time and without use or, respectively, loading of the MR computer 100′, i.e. without a computing time of the processor 110 and without an access time of the working memory 120. The raw MR data are thus removed from the MR computer 100′, and said MR computer 100′ can continue to calculate or reconstruct MR images that are required for an immediate assessment (for example within the scope of an early monitoring) with a higher priority. A data volume of the MR data can be very large, for example in a comprehensive MR measurement.

A qualitatively high-grade MR image is calculated subsequently (or in the meanwhile) as a final MR image using a complicated calculation method or reconstruction method, for example for diagnosis. The image calculation or image reconstruction using the complicated calculation method, or reconstruction method, takes place via the computer 500, to which the raw MR data stored in MR memory 400 are provided, and the resulting calculation time is dependent on the computing power of the computer 500. The computer 500 can require a significantly longer computing time (in the range of a few minutes to multiple hours) for the image calculation or, respectively, image reconstruction using the complicated calculation method or, respectively, reconstruction method for the qualitatively high-grade MR image, even if the computer 500 is executed as a very high-performance computer with very fast processor 510, wherein the MR system 20 (and in particular the MR computer 100′) is already provided for additional MR measurements.

The computer 500 can be designed as a powerful workstation or virtual machine and/or be outsourced with other computers to a server or the cloud. Since the MR data are cached in MR memory 400, they can be transferred into the cloud and processed there without time pressure. Since the MR images calculated or reconstructed in the cloud are not required for an immediate assessment (for example within the scope of the early monitoring), these MR images can be calculated or reconstructed in the cloud with a lower priority with acceptance of a longer transfer time for the raw MR data.

The calculation method or reconstruction method can be distributed to multiple computers. Multiple computers can execute multiple calculation methods or reconstruction methods (for example for MR data of multiple MR measurements) simultaneously at least in part.

The qualitatively high-grade MR image can be stored in the memory 480 of the MR memory 400.

The stored, qualitatively high-grade MR image can be processed further (for example displayed) in the MR computer 100′ to which the qualitatively high-grade MR image stored in the MR memory 400 is provided.

Within the scope of a retrospective reconstruction, the MR system 20 can thus calculate the qualitatively high-grade MR image from the raw MR data that have been cached outside of the MR computer 100′ during the MR measurement using the high-performance calculation method outside of said MR computer 100′.

In a further embodiment, the MR computer 100′ can include the memory 400. In another embodiment, the memory 400 and the MR computer 100′ can be realized in one device.

In a further embodiment, the computer 500 can comprise the memory 400. In another embodiment, the memory 400 and the computer 500 can be realized in one device.

Although modifications and changes may be suggested by those skilled in the art, it is the intention of the inventor to embody within the patent warranted hereon all changes and modifications as reasonably and properly come within the scope of his contribution to the art.

I claim as my invention:

1. A medical imaging system comprising:
a data acquisition unit;
a first computer configured to operate said data acquisition unit to acquire raw data from a patient interacting with said data acquisition unit;
a memory in communication with said first computer in which said raw data are stored; and
a second computer, different from said first computer, having access to said memory and configured to access the raw data stored in said memory and to calculate an image of the patient from the accessed raw data.

2. An imaging system as claimed in claim 1 comprising a network providing a communication path between said memory and said second computer via which said raw data is transferred from said memory to said second computer.

3. An imaging system as claimed in claim 1 wherein said second computer is selected from the group consisting of a workstation, a virtual machine, and a cloud computer.

4. An imaging system as claimed in claim 1 wherein said second computer is configured to calculate said image using a calculation algorithm that is not executable by said first computer.

5. An imaging system as claimed in claim 4 wherein said image is a first image, and wherein said first computer is configured to calculate a second image from said raw data by executing an image calculation algorithm that is less computationally intensive than said algorithm executed by said second computer to calculate said first image.

6. An imaging system as claimed in claim 1 wherein said memory is located at a location selected from a location connected to said first computer, a location in said first computer, and a location in said second computer.

7. A computerized method for operating an imaging system, comprising:
from a first computer, controlling an image data acquisition device to acquire raw data from a patient;
storing the acquired raw data in a memory; and
from a second computer, accessing the raw data stored in the memory and calculating, in said second computer, an image of the patient from the raw data accessed from the memory.

8. A method as claimed in claim 7 comprising transferring the raw data from the memory to the second computer via a network forming a communication path between the memory and the second computer.

9. A non-transitory, computer-readable data storage medium encoded with programming instructions, said storage medium being distributively loaded in a first computer and a second computer of an imaging system that also comprises a data acquisition device, and said programming instructions causing:
said first computer to operate said data acquisition device to acquire raw data from a patient in the data acquisition device;
transfer the acquired raw data to a memory for storage therein; and
the second computer to access the raw data from the memory and to calculate an image of the patient from the accessed raw data.
